(19) United States
(12) Patent Application Publication
(10) Pub. No.: US 2003/0219502 A1
(43) Pub. Date: Nov. 27, 2003

(54) Plaster of paris fabric

(76) Inventors: Andreas Radspieler, Gieboldehausen (DE); Karl Becker, Duderstadt (DE)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/400,912

(22) Filed: Mar. 28, 2003

(30) Foreign Application Priority Data
Apr. 11, 2002 (DE) 202 05 577.9

(51) Int. Cl. A61F 2/80; A61F 5/01

(52) U.S. Cl. 425/2; 623/33; 264/222

(57) ABSTRACT

Plaster of Paris fabric, in particular for producing a negative impression of a body part, consisting of a textile fabric with plaster of Paris powder scattered over it and pressed into it, characterized in that the textile fabric is configured as a tube.

The invention relates to a plaster of Paris fabric, in particular for producing a negative impression of a body part, consisting of a textile fabric with plaster of Paris powder scattered over it and pressed into it.

Plaster of Paris fabrics of this kind are sold as plaster of Paris bandages which are rolled up in a circle and available in different lengths. To use it, the rolled-up plaster of Paris bandage is dipped in water and then wound around the body part of which a negative impression is to be made, for adaptation of prostheses or ortheses. After the plaster has set, the plaster cast is removed and the negative impression is ready.

Plaster of Paris bandages of this kind are also used to immobilize limbs with broken bones.

A disadvantage of these known plaster of Paris bandages is that the winding procedure results in poor reproducibility when a later second impression is taken. This is attributable, on the one hand, to the different winding techniques used by different practitioners involved in producing negative impressions and, on the other hand, to different tensioning within the fabric of the plaster of Paris bandage.

A further disadvantage is that the individual winding technique results in different numbers of layers being wound, which fact subsequently leads to different wall thicknesses in the negative impression and makes further processing more difficult, for example if individual parts have to be cut out from the impression.

Starting out from these problems, the plaster of Paris fabric described at the beginning is to be improved in such a way that a reproducible impression can be produced, so as to obtain a wall thickness which is as uniform as possible.

To solve the problem, a plaster of Paris fabric of the generic type is characterized in that the textile fabric is configured as a tube.

By means of this configuration, a one-layer bandage is created which is simply pulled onto the body part. The negative impression is therefore produced not by winding, but by compression-free shaping, without the outer configuration being able to be affected. In this way, a negative impression can be made which is always reproducible and has the same wall thicknesses. From the tube, a plaster of Paris stocking is obtained whose use and shaping are very easily managed.

If the textile fabric is made thick enough, the plaster of Paris fabric can also be used to medically treat broken limbs.

The tube can be closed at one end. This is preferably done if a negative impression of an arm stump or leg stump is to be made for adaptation/production of a prosthesis.

The textile fabric is preferably partly elastic. This ensures that, within a certain range, negative impressions of limbs of different thickness can be prepared. In particular, the partial elasticity preferably results only from the slack in the meshes obtained during weaving. The textile fabric can also be made fully elastic, by which means a greater range of limbs of different thickness can be covered.

It is particularly advantageous if the tube can be rolled up to form a ring. The dry plaster of Paris fabric can then simply be rolled up onto the limb after dipping in water, which further simplifies handling.

To produce the fabric tube or fabric stocking, the textile fabric is for example drawn onto a cylindrical drum, covered with plaster like a conventional plaster of Paris bandage, and this plaster is then pressed into the fabric while the cylindrical drum is rotating.

A cloth-like fabric can likewise be scattered with plaster and pressed, and this is then sewn together to form a tube. The fabric has a thickness of preferably 1 to 10 mm and the consistency of a known terry towel.

1. Plaster of Paris fabric, in particular for producing a negative impression of a body part, consisting of a textile fabric with plaster of Paris powder scattered over it and pressed into it, characterized in that the textile fabric is configured as a tube.

2. Plaster of Paris fabric according to claim 1, characterized in that the tube is closed at one end.

3. Plaster of Paris fabric according to claim 1, characterized in that the textile fabric is partly elastic.

4. Plaster of Paris fabric according to claim 1 or 2, characterized in that the tube can be rolled up to form a ring.
